(19) United States
(12) Patent Application Publication
(10) Pub. No.: US 2025/0177641 A1
(43) Pub. Date: Jun. 5, 2025

(54) MEDICATION DELIVERY DEVICE

(71) Applicant: Becton, Dickinson and Company, Franklin Lakes, NJ (US)

(72) Inventors: Andrew BEAUPRE, Somerville, MA (US); Ronald MOULTON, Woburn, MA (US); Jesse KAUFFMAN, Andover, MA (US); Scott STEWART, Somerville, MA (US); Alexander SOKOLOV, Woodbury, NY (US); Ralph CASSARA, Massapequa, NY (US); Karl KEPPELER, Manorville, NY (US); Jan NIEWIADOMSKI, Aquebogue, NY (US); Clint BROWN, Port Jefferson, NY (US); Roy WANG, Miller Place, NY (US)

(73) Assignee: Becton, Dickinson and Company, Franklin Lakes, NJ (US)

(21) Appl. No.: 18/844,860

(22) PCT Filed: Mar. 8, 2023
(86) PCT No.: PCT/US2023/014855

§ 371 Date: Sep. 6, 2024

Related U.S. Application Data
(60) Provisional application No. 63/318,258, filed on Mar. 9, 2022.

(51) Int. Cl. A61M 5/142

(52) U.S. Cl. CPC A61M 5/14216; A61M 2205/3306; A61M 2205/3317; A61M 2205/3365; A61M 2205/3379

(57) ABSTRACT

A medication delivery device comprising a base plate engaging a cover to form an interior, the interior including a pump drive mechanism for driving medication through the device, a drivetrain engaged to the pump drive mechanism, a magnetic encoder including an encoder hub mechanically coupled to the drivetrain, the encoder hub including a magnet, and a sensor that measures a magnetic field from the magnet to determine a rotational position of the drivetrain.

### CROSS-REFERENCE

This application claims priority under 35 U.S.C. 119 (e) to U.S. provisional application Ser. No. 63/318,258, filed on Mar. 9, 2022, which is incorporated herein by reference in its entirety.

### FIELD

The present disclosure relates to medical devices, and more particularly to medication delivery devices with an infusion pump system capable of detecting an amount of medication dispensed.

### BACKGROUND

Infusion pumps generally have a reservoir with a known volume of medication and known dispense stroke volume to count down doses and estimate how much medication is expelled from the reservoir, out of the infusion pump and into the patient. Alternative forms of encoding to determine the amount of medication expelled include mechanical switches and spring loaded electrical connection.

For example, two electrically conductive spring elements and electrically conductive material coupled with a drive gear can be used to sense angular rotation. The electrically conductive material coupled to the drive gear is spaced out so that a first spring contacts either an insulator or the electrically conductive material based on the rotational position of the drive gear. A second spring is always contacting electrically conductive material. Thus, when the drive gear rotates, the electrically conductive springs intermittently complete, or short, the circuit. The number of increments between shorts confirms the drive gear rotation and ultimately the amount of medication dispensed.

For medical devices such as a wearable medication delivery pump, a patch pump or an infusion pump, other means to measure how much medication is expelled is desired. In general, a need exists for accurate detection of medication delivery that improves accuracy, product life, energy efficiency and manufacturability, while reducing part count and cost.

### SUMMARY

Exemplary embodiments disclosed herein may provide alternative solutions at least the above problems and/or disadvantages, as well as others not described above. Also, exemplary embodiments are not required to overcome, and may not overcome, the disadvantages, if any, described above.

The matter exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the disclosure. Also, descriptions of well-known functions and constructions are omitted for clarity and conciseness.

An exemplary aspect of the present disclosure provides a low resolution magnetic encoder in a medication delivery device in which a series of magnets are fixed to an encoder hub and mechanically coupled to a drive gear. The drive gear can be aligned to a magnetic Hall Effect sensor to provide rotational feedback indicative of a detected magnetic field.

Another exemplary aspect of the present disclosure provides a low resolution magnetic encoder comprising a single multi-pole magnet in a medication delivery device that can be mechanically coupled to a drive gear, whereby the drive gear can be aligned to a magnetic Hall Effect sensor to provide rotational feedback indicative of a detected magnetic field.

Exemplary implementations of disclosed aspects can be used to measure the rotational position of a drive gear of a drivetrain to facilitate detection of the amount of medication dispensed from a medication delivery device.

According to exemplary implementations, magnetic Hall Effect sensor can be configured so as not to increase friction acting on the drive gear. As a result, according to exemplary implementations, the drive train can be more power efficient due to less external physical impact.

According to further exemplary implementations, the resolution of a magnetic encoder can be varied by increasing or decreasing the number of magnetic poles observed within a given angular rotation by a magnetic Hall Effect sensor. For example, a magnetic encoder can be optimized by maximizing the number of magnetic poles while avoiding magnetic interference between the magnetic poles or cross talk between the magnets.

According to exemplary implementations, a single multi-pole magnet can be configured to provide a low part count solution, increase manufacturability, and reduce cost of the magnetic encoder solution via a simpler production process.

In yet further exemplary implementations, an optical sensor can be provided in conjunction with a magnetic encoder, for example to facilitate finer motor control and higher medication delivery accuracy.

Exemplary embodiments of the present disclosure provides a medication delivery device comprising a base plate engaging a cover to form an interior, the interior including a pump drive mechanism for driving medication through the device, a drivetrain engaged to the pump drive mechanism, an encoder hub mechanically coupled to the drivetrain, the encoder hub including a magnet, and a sensor that measures a magnetic field from the magnet to determine a rotational position of the drivetrain.

In accordance with a further example aspect of the present disclosure, the medication delivery device can include one of a patch pump, an infusion pump and an automated insulin delivery device.

In accordance with a further example aspect of the present disclosure, the pump drive mechanism can include a plunger driven by a leadscrew.

In accordance with a further example aspect of the present disclosure, the drivetrain can include a drive gear, an output of the drivetrain engaging a drive nut, and the drive nut being a rotating component that engages the leadscrew.

In accordance with a further example aspect of the present disclosure, the drive gear can be aligned with the sensor.

In accordance with a further example aspect of the present disclosure, the encoder hub can be coupled to the drive gear to measure the rotational position.

In accordance with a further example aspect of the present disclosure, the magnet can be configured or implemented as a plurality of magnets, for example equally spaced.

In accordance with a further example aspect of the present disclosure, a configuration can be provided where polarity of a plurality of magnets facing away from a center axis of the drive gear remains constant.

In accordance with a further example aspect of the present disclosure, a configuration can be provided where polarity of a plurality of magnets facing away from a center axis of the drive gear alternates in series with respect to the drive gear.

In accordance with a further example aspect of the present disclosure, a configuration can be provided where the magnet can be axially polarized.

In accordance with a further example aspect of the present disclosure, a configuration can be provided where the magnet can be radially polarized.

In accordance with a further example aspect of the present disclosure, the magnet can be configured as a single multi-pole magnet with a plurality of magnet arms spaced around the encoder hub.

In accordance with a further example aspect of the present disclosure, a configuration can be provided where a sensor can be mounted on a top surface of the circuit board.

In accordance with a further example aspect of the present disclosure, a configuration can be provided where a sensor can be mounted on a bottom surface of the circuit board.

In accordance with a further example aspect of the present disclosure, a configuration can be provided where a sensor can be aligned vertically with an axis of rotation of the drive gear.

In accordance with a further example aspect of the present disclosure, the sensor can include one of a magnetic Hall Effect sensor and a magnetic resistance sensor.

In accordance with a further example aspect of the present disclosure, the medication delivery device can include an optical sensor, for example, disposed in a middle portion of the drivetrain.

In accordance with a further example aspect of the present disclosure, the optical sensor can include an emitter that emits a light from an LED or a laser light, for example, and a receiver that detects the light or the laser light.

In accordance with a further example aspect of the present disclosure, the optical sensor can be configured to include optical fan blades configured to split the light, laser light or beam, the optical sensor can also be configured to detect when the light, laser light or beam is split.

In accordance with a further example aspect of the present disclosure, the magnetic encoder can be configured to include a ring magnet having one or more magnetized sections.

Additional and/or other aspects and advantages of the present disclosure will be set forth in the description that follows, or will be apparent from the description, or may be learned by practice of the disclosure.

### BRIEF DESCRIPTION OF THE DRAWINGS

The above and/or other example aspects and advantages of the present disclosure will become apparent and more readily appreciated from the following description of example embodiments, taken in conjunction with the accompanying drawings in which:

FIG. 1 is a perspective view of a wearable medication delivery device constructed in accordance with an exemplary embodiment of the present disclosure;

FIG. 2 is a top perspective view of the medication delivery device of FIG. 1, with the cover removed and constructed in accordance with an exemplary embodiment of the present disclosure;

FIG. 3 is a block diagram of exemplary components of a medication delivery device of FIG. 1 constructed in accordance with an exemplary embodiment of the present disclosure;

FIG. 4 is a side view of a drivetrain of the medication delivery device of FIG. 2 constructed in accordance with an exemplary embodiment of the present disclosure;

FIG. 5 is a perspective view of the motor and planetary gearbox of the medication delivery device of FIG. 4 constructed in accordance with an exemplary embodiment of the present disclosure;

FIG. 6 is a side view of magnetic poles in a magnetic encoder of the medication delivery device of FIG. 2 constructed in accordance with an exemplary embodiment of the present disclosure;

FIG. 7 is a legend and a graph identifying the magnetic hall effect sensor output based on the magnetic poles in the magnetic encoder of the medication delivery device of FIG. 6 constructed in accordance with an exemplary embodiment of the present disclosure;

FIG. 8 is a graph presenting sensor measurements for the magnetic encoder with eight poles in the medication delivery device of FIG. 6 constructed in accordance with an exemplary embodiment of the present disclosure;

FIG. 9 is a side view of eight magnetic poles in the magnetic encoder of the medication delivery of FIG. 6 constructed in accordance with an exemplary embodiment of the present disclosure;

FIG. 10 is a perspective view of a ring magnet in a magnetic encoder disposed in a medication delivery device constructed in accordance with a second exemplary embodiment of the present disclosure;

FIG. 11 is a cross-sectional view of a multi-pole magnetic encoder disposed in a medication delivery device constructed in accordance with a third exemplary embodiment of the present disclosure;

FIG. 12 is a graph presenting sensor measurements for a magnetic encoder with twenty poles in the medication delivery device of FIG. 11 constructed in accordance with an exemplary embodiment of the present disclosure; and

FIG. 13 is a graph presenting sensor measurements for the multi-pole magnetic encoder with twenty poles including missed interrupts in a medication delivery constructed in accordance with a fourth exemplary embodiment of the present disclosure.

Throughout the drawing figures, like reference numbers will be understood to refer to like elements, features, and structures.

### DETAILED DESCRIPTION OF EXEMPLARY EMBODIMENTS

Referring now to the drawings, wherein like reference numerals designate identical or corresponding parts throughout the several views, embodiments of the present disclosure are described as follows.

It will be understood that the terms “include,” “including,” “comprise,” and/or “comprising,” when used in this specification, specify the presence of stated features, integers, steps, operations, elements, and/or components, but do not preclude the presence or addition of one more other features, integers, steps, operations, elements, components, and/or groups thereof.

It will be further understood that, although the terms, “first,” “second,” “third,” etc., may be used herein to describe various elements, components, regions, layers and/or sections, these elements, components, regions, layers and/or sections may not be limited by these terms. These terms are only used to distinguish over element, component, region, layer or section from another element, component, region, layer or section.

As used herein, the term “and/or” includes any and all combinations of one or more of the associated listed items. Expressions such as “at least one of,” when preceding a list of elements, modify the entire list of elements and do not modify the individual elements of the list. In addition, the terms such as “unit,”-er (-or),” and “module” described in the specification refer to an element for performing at least one function or operation, and may be implemented in hardware.

Various terms are used to refer to particular system components. Different companies may refer to a component by different names—this document does not intend to distinguish between components that differ in name but not function.

Matter of these exemplary embodiments that are obvious to those of ordinary skill in the technical field to which these exemplary embodiments pertain may not be described here in detail. In addition, various features of the exemplary embodiments can be implemented individually or in any combination or combinations, and would be understood by one of ordinary skill in the art of medicament delivery devices.

The example embodiments of a low resolution magnetic encoder in a medication delivery device as described below are useful to accurately measure dispensed medicament. The medication delivery device includes, for example, a patch pump, an infusion pump or an automated insulin delivery device. The infusion pump system is generally understood to be a type of pump that works on the principle of filling a chamber (for example, with liquid medication from a reservoir) in one stage and then emptying the medication from the chamber (for example, to a delivery device such as a cannula deployed in a patient) in another stage.

In the exemplary embodiment of the present disclosure, further, the low resolution magnetic encoder described below employs a number of technical principles such as: (a) a magnetic hall effect sensor; (b) a series of magnets that provide configurable magnetic poles; (c) an optical sensor that provides fine motor control and high medication delivery accuracy; and (d) a multi-pole magnetic encoder or ring magnet design.

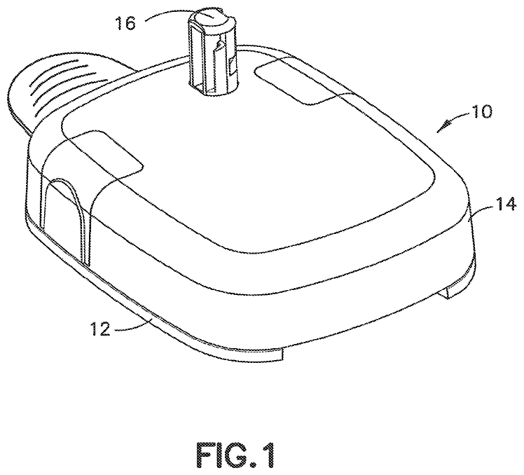

FIG. 1 is a perspective view of the wearable medication delivery device 10 including a baseplate 12, a cover 14, and an insertion mechanism 16. The insertion mechanism 16 is configured to insert a cannula into a skin of the patient to deliver a medication.

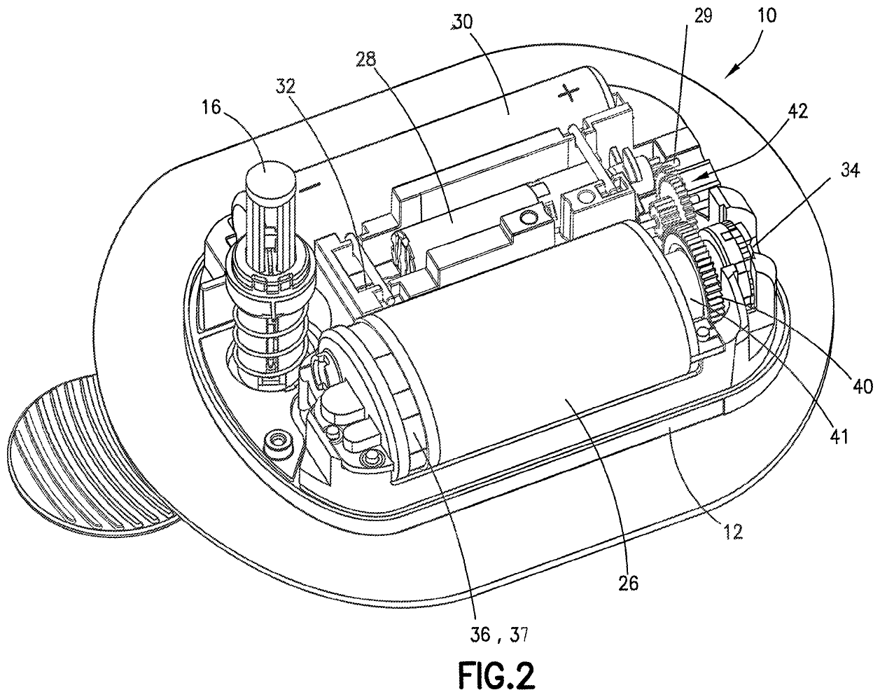

FIG. 2 is a perspective view of the medication delivery device 10 of FIG. 1 with the cover 14 removed. The base plate 12 supports the insertion mechanism 16, a motor 28, a power source such as a battery 30, a printed circuit board (PCB) 32, a memory 33, and other electronic components, and a reservoir 26 for storing the medication to be delivered to the patient (or user) via an outlet fluid path from an outlet port of the reservoir 26 through the insertion mechanism 16. In an exemplary implementation, the reservoir 26 has an inlet port connected to the fill port via the inlet fluid path to receive the medication.

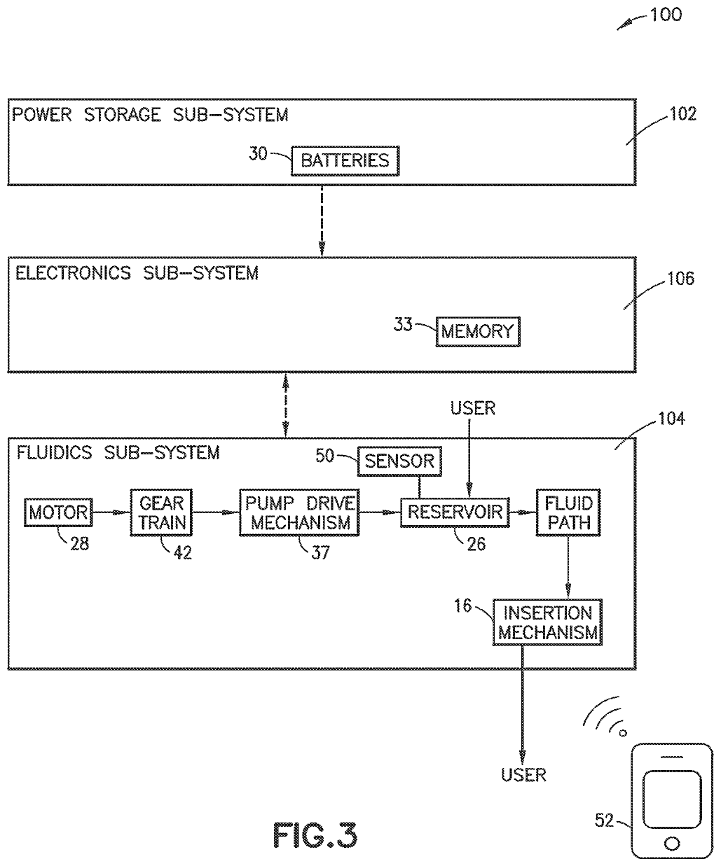

FIG. 3 is a system diagram 100 that illustrates an example implementation of components in an example medication delivery device 10 having an infusion pump system of FIGS. 1-3, for detecting the delivered medication. Referring to the example of FIG. 3, the medication delivery device 10 can include a power storage sub-system 102, a fluidics sub-system 104, and an electronics sub-system 106 for controlling operations of components in the fluidics sub-system 104 such as the insertion mechanism 16 for deploying the cannula for inserting into an infusion site on a patient's skin. The power storage sub-system 102 includes a power source, such as one or more batteries 30, for example, for providing power to components in the electronics and fluidics sub-systems 104 and 106. The fluidics sub-system 104 includes a motor 28, a drivetrain 42, a pump drive mechanism 37 and the reservoir 26 filled with a medication. The pump drive mechanism 37 includes a plunger 36 driven by a leadscrew 34. Further, the electronics sub-system 106 includes a memory device 33 cooperating with a magnetic Hall Effect sensor 50 to measure medication dispensed.

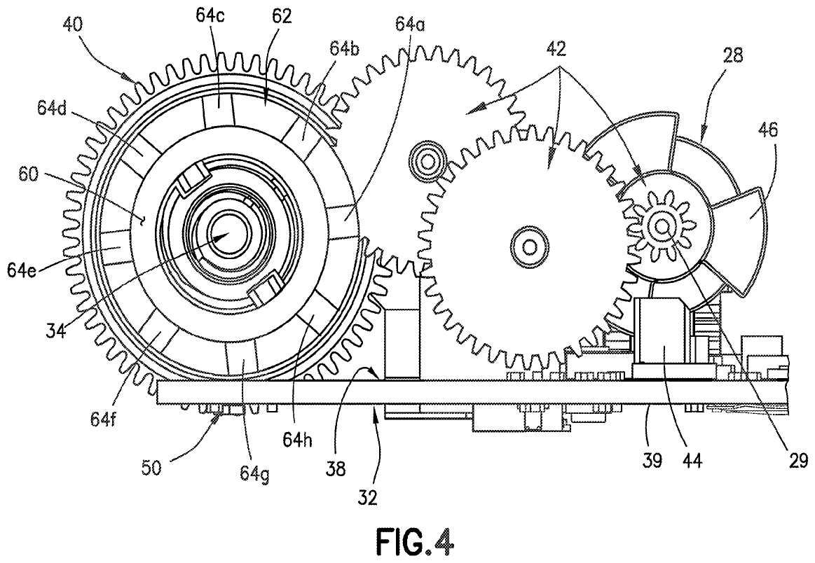

FIG. 4 illustrates a drivetrain 42 cooperating with a magnetic encoder 60. The drivetrain 42 is illustrated as a compound gear, but one of ordinary skill in the art would readily appreciate that other components can be implemented to translate input rotation of motor 28 to a drive gear 40 and a drive nut (or leadscrew nut) 41. For example, current can be applied to rotate the motor 28 and translate this rotation throughout the drivetrain 42 to the drive gear 40. The drive nut 41 is advantageously rotated by the drive gear 40. In an exemplary implementation, the drive nut 41 is the rotating component that engages, or can be rotationally coupled with, the leadscrew 34. Rotation of the leadscrew 34 causes linear motion of the plunger 36 and ultimate ejection of the medication from the medication delivery device 10. In other words, in an exemplary implementation, the drive nut 41 is the mechanically closest rotating component to the plunger 36 whereby detection of rotational motion, or lack thereof, of the drive nut 41 provides a proximate indication of axial displacement, or lack thereof, of plunger 36. In an exemplary implementation of FIG. 4, the drive gear 40 is rotationally fixed to the drive nut 41, and engaged with the drivetrain 42 operated by the motor 28. Further, the drive gear 40 is constrained by the leadscrew 34 and a main frame of the assembly to react to forces from a plunger movement and from fluid pressure.

In another embodiment, the motor 28 includes an output shaft 29 that is in line with and directly drives the pump drive mechanism 37 without the use of a drivetrain 42 in a similar manner as described above. An exemplary motor 28 includes a stepper motor. The stepper motor could be used if the form was different (for example, a narrow reservoir 26). In other words, there would likely be a significant loss of resolution unless a small pitch of the leadscrew 34 was implemented (difficult to achieve) or a cross section of the reservoir 26 is decreased. A smaller cross section of the reservoir 26 is much easier to achieve especially when delivering a small volume of medication.

In this embodiment, the resolution of the magnetic encoder 60 can be measured at any place in the drivetrain 42 based on the desired tradeoff between the precision of sensor 50 and the required resolution. For example, the resolution can be improved if the sensor 50 in the drivetrain is moved closer to the motor. However, a more precise sensor 50 would be required to achieve the improved resolution. The sensor 50 can be positioned at an output of the drivetrain 42 where the low resolution encoder is measured. Alternately, the sensor 50 can be positioned at an input end of the drivetrain 42 where the high resolution encoder is measured.

In yet another embodiment, the drivetrain 42 could include a belt system, friction gears or other forms of power transmission (as opposed to traditional gears). Friction gears could be advantageous if there are backlash-type problems with traditional gears.

FIG. 4 further illustrates the printed circuit board 32 having a top surface 38 and a bottom surface 39 with a sensor 50 disposed on one of the two surfaces. The sensor 50 includes a low resolution magnetic proximity sensor such as a magnetic Hall Effect sensor or a magnetic resistance sensor. Various magnetic Hall Effect sensors 50 can be used to accommodate different magnetic field strengths and/or sensitivities.

In an exemplary embodiment, the sensor 50 can be disposed anywhere in an interior of the medication delivery device 10, i.e., between the cover 14 and the base plate 12. In another embodiment, the sensor 50 can be connected by a wire or a flexible circuit, instead of via the circuit board 32.

In an exemplary implementation, sensor 50 can be aligned vertically to an axis of rotation of the drive gear 40 to measure a magnetic field of a magnet 64 and thereby confirm any rotation of the drive gear 40 for a given rotational input from the motor 28, where the rotation of drive gear 40 would result in a corresponding movement of plunger 26. The output of sensor 50 can thus be used to provide real-time feedback of medication being dispensed, for example, via a Bluetooth device to a smart phone 52. In another exemplary implementation, the output of sensor 50 can facilitate accurate control of the dispensing operation of the medication delivery device 10. According to exemplary configurations, sensor 50 can be positioned at a predetermined distance from the magnet 64 to increase or decrease sensitivity of the magnetic field measurement. In a preferred embodiment, the sensor 50 is attached to the pump drive mechanism 37. Further operation of the sensor 50 is described below.

FIGS. 4-7 illustrate an exemplary configuration of the magnetic encoder 60 cooperating with the drivetrain 42 and the sensor 50. The magnetic encoder 60 includes an encoder hub 62, for example, configured on or integrated with a drive gear 40, and the magnet member 64, for example, including multiple magnets 64a, 64b, 64c, 64d, 64e, 64f, 64g, 64h configured on encoder or integrated with hub 62, creating a magnetic field. During operation, the magnet member 64 rotates with drive gear 40 and as a magnet passes over the Hall Effect sensor 50, an interrupt measured by sensor 50 is triggered. The interrupt aids in determining a rotational position of the drive gear 40 and the drivetrain 42 as measured by the Hall Effect sensor 50 and facilitates monitoring of accurate operation of the drive gear 40 and the drivetrain 42.

In an exemplary implementation, encoder hub 62 is mechanically coupled to the drive gear 40, which rotationally advances the leadscrew 34. Accordingly, output of Hall Effect sensor 50 based on rotation of encoder hub 62 can facilitate measuring or monitoring the rotational position of drive gear 40, whereby implementation of a magnetic encoder 60 can ensure continuity of the drivetrain 42. For example, if the number of interrupts measured by sensor 50 do not match an expected amount, for example, with a certain time period, the magnetic encoder 60 can trigger an alarm. The alarm notifies the user that the motor 28 and the magnetic encoder 60, and therefore drive gear 40, are sufficiently decoupled, for example due to lack of continuity of drivetrain 42. In an exemplary implementation, such feedback of encoder 60 can be used, for example, to determine under rotation and/or over rotation of the drive gear 40, as an indication of under dosing and over dosing of medicament to the patient due to corresponding axial movement of plunger 36.

In an exemplary implementation, magnetic material of the magnet member 64 can be changed to provide different magnetic field strengths (N52 or N42, for example). Exemplary base materials of magnets that can be used include neodymium and iron oxide, although other magnet materials are contemplated as understood by one skilled in the art. In an exemplary implementation, the magnet member 64 includes one or more magnets 64a, 64b, 64c, 64d, 64e, 64f, 64g, 64h, etc. disposed on the encoder hub 62 which is coupled to the drive gear 40. When two or more magnets 64a, 64b, 64c, 64d, 64e, 64f, 64g, 64h, etc. are configured equidistantly from each other and at the same distance from central axis of drive gear 40, an expected number of interrupts can be observed by the Hall Effect sensor 50 over one full rotation of the drive gear 40, which in turn is based on the input rotation of the motor 28 via drivetrain 42.

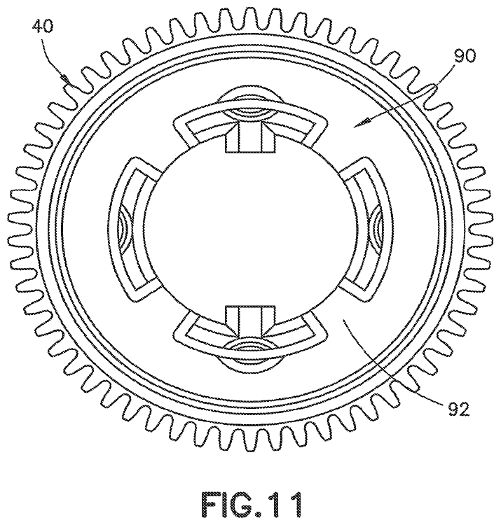

In another exemplary implementation of disclosed embodiments, as illustrated for example, in FIG. 11, the magnetic encoder 60 includes a single multi-pole magnet 90 with a plurality of magnet arms 92 equally spaced. In an exemplary implementation of disclosed embodiment, a single multi-pole magnet 90 can replace magnet 64 and encoder hub 62 of the magnetic encoder 60. For example, a multi-pole magnet 90 can be molded as one component and then magnetized during a separate processing step. Both of these configurations are described below.

The plurality of magnets 64a, 64b, 64c, 64d, 64e, 64f, 64g, 64h, etc. can be arranged in a variety of polarities. In an exemplary implementation of disclosed embodiments, the polarity of the plurality of magnets 64a, 64b, 64c, 64d, 64e, 64f, 64g, 64h, etc. facing away from a center axis of the drive gear 40 remains constant. In another exemplary embodiment, the polarity of the plurality of magnets 64a, 64b, 64c, 64d, 64e, 64f, 64g, 64h, etc. facing away from a center axis of the drive gear 40 alternates in series with respect to the drive gear 40. In yet another exemplary embodiment, the polarity of the plurality of magnets facing away from a center axis of the drive gear 40 does not alternate in series with respect to the center axis of drive gear 40. For example, the polarity is randomly arranged with respect to center axis of the drive gear 40. Further, the plurality of magnets 64a, 64b, 64c, 64d, 64e, 64f, 64g, 64h, etc. can be radially polarized as illustrated in FIGS. 2, 9 and 10. According to still another exemplary embodiment, the plurality of magnets 64a, 64b, 64c, 64d, 64e, 64f, 64g, 64h, etc. can be configured to be axially polarized. For example, the magnetic pole of each of a pair of magnets 64 can include, for example, one of NNNN, NSNS, SNNN, or NNSS. The above configurations are exemplary and one skilled in the art would appreciate other pole orientations, number of magnets used, polarity patterns and various hub materials, for example, within the scope of the present disclosure.

FIG. 4 further illustrates an exemplary embodiment including optical sensor 44 and optical fan blades 46. The optical sensor 44 can be configured to include an emitter and a receiver, for example of a laser beam, with the optical fan blades 46 being disposed between the emitter and the receiver. The optical sensor 44 identifies an interrupt when the receiver determines that one of the optical fan blades 46 rotates and blocks the emitted laser beam, or splits the emitted laser beam, such that, for example, the optical sensor 44 receives a binary feedback based on whether the laser beam is split.

In another embodiment, the optical sensor 44 includes a light emitted from a light emitting diode (LED). The optical fan blades 46 would operate with the light similarly to the laser beam as described above.

In an exemplary implementation, optical fan blades can include three optical fan blades 46 spanning 60° whereby the gap between each of the optical fan blades 46 is also 60°. Such a configuration provides six transition points for the laser beam for every 360° of rotation of the optical fan blades 46. The transition points are defined where the laser beam transitions from contacting the optical fan blade 46 to being received by the receiver and when the laser beam transitions from being received by the receiver to contacting the optical fan blade 46 (vice versa). In various exemplary implementations, more blades can be provided in conjunction with the optical sensor 44. In certain exemplary configurations, varying dimensions, number, and positioning of fan blades can increase or reduce the error rate of detection by optical sensor 44.

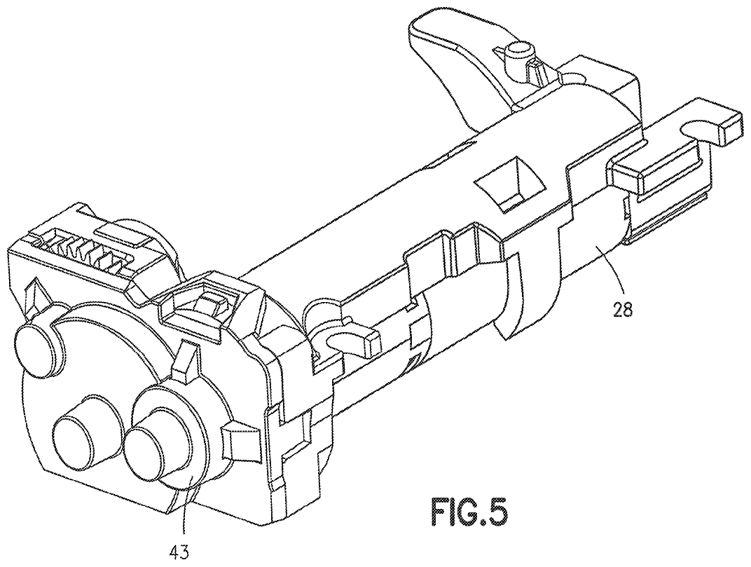

With reference to FIG. 5 an exemplary configuration provides motor 28 including a planetary gearbox 43 at the output of the motor 28 with optical fan blades 46 of the optical sensor 44 coupled to the output of the planetary gearbox 43. As illustrated in FIGS. 4 and 5, the drivetrain 42 includes, or mechanically couples with, the gears in the planetary gearbox 43. Thus, the optical sensor 44 is can be positioned essentially in the middle of the drivetrain 42 to provide unique benefits while balancing various design objectives as described below, where for example, gear reduction or gear amplification can take place upstream and downstream from the optical sensor 44.

In an exemplary implementation, the accuracy of detection and monitoring of drive gear 40 rotation can be maximized despite requiring very small angular rotation. For example, placing the optical sensor 44 closer to an output shaft 29 of the motor 28 allows the gear reduction from the drivetrain 42 to amplify and increase the resolution to provide more accurate measurement of very small angular rotation (to allow for a finer control of the input rotation). According to another example, disposing the optical sensor 44 on the drive gear 40, may result in a more coarse control or a small degree of angular rotation (1.28°, for example) at the drive gear 40.

According to further exemplary implementations, the optical sensor 44 can minimize the potential for gear windup or backlash when positioning the optical sensor 44 closer to the output of the drivetrain 42. In this manner, the amplification of gear tooth error and error from elastic or plastic deformation in the drivetrain 42 can be minimized. For example, if the optical sensor 44 were to engage the back of the motor 28 and before any gear reduction, rotational error can accumulate throughout the full drivetrain 42. Tolerance clearance in the gears of the drivetrain 42 (gear backlash) can also slightly reduce output rotation per a given input rotation of the motor 28, as well as the mechanical fits (windup due to elastic or plastic deformation) of the components.

In an exemplary implementation, optical sensor 44 can measure the angular rotation of the motor 28 to accurately determine the linear output of the plunger 36 and thus the delivery of medication from the medication delivery device 10. For example, when the optical sensor 44 detects a transition during the rotation of the optical fan blades 46 as described above, the current can be cut and the motor 28 would stop operation to indicate that the dose administration is complete. The motor 28 can then resume operation when the next dose is ready to be administered and subsequently the motor 28 can again be stopped when a transition is detected to indicate that the dose administration is complete. This process can be repeated until all doses are administered. As described in the above examples, the medication delivery device 10 according to exemplary embodiments, can be configured to provide variable dosage rates at either a consistent amount of time between doses or a variable amount of time between doses.

As described in the above examples, the location of the optical sensor 44 and the optical fan blades 46 can be optimally selected with respect to the drivetrain 42, drive gear 40, motor 28, and/or gearbox 43. In exemplary implementations where the optical sensor 44 is positioned essentially in the middle of the drivetrain 42, both resolution and error are split and a compromise can be achieved to meet various design objectives. Such an exemplary configuration can be more cost and weight effective because optical sensors 44 can be relatively inexpensive and may not require an increase in the size and/or weight of the medication delivery device 10. Thus, the use of optical sensors 44 as disclosed herein can provide advantageous size and weight savings to the medication delivery device 10.

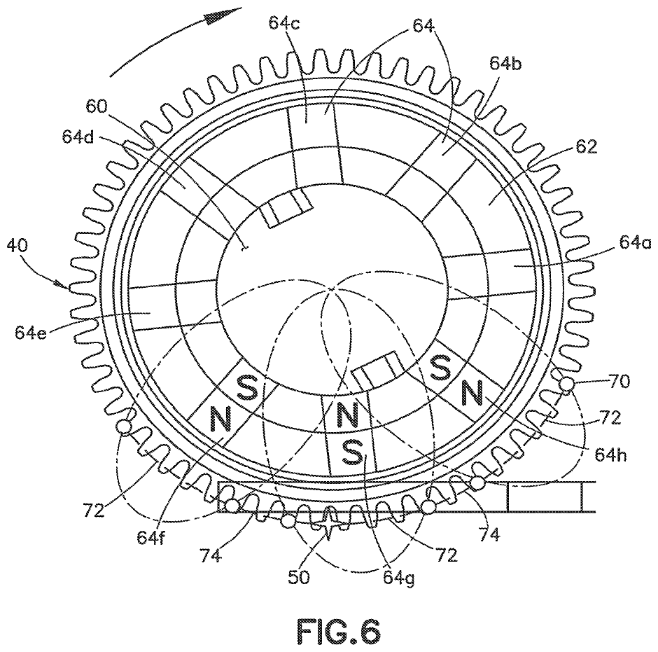

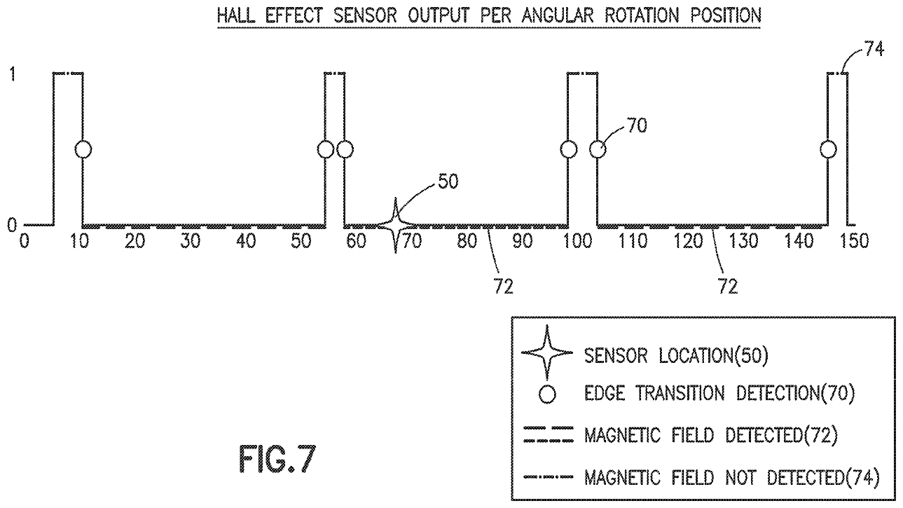

According to exemplary embodiment, as illustrated in the example of FIGS. 6 and 7, sensor 50 can be implemented and cooperate with the magnetic encoder 60. In an exemplary implementation, the sensor 50 can be located at approximately an outer edge of one of the teeth of the drive gear 40 in a vertical direction from the center of the drive gear 40 and the magnetic encoder 60. When one tooth to the next tooth of the drive gear 40 moves with respect to sensor 50, an edge transition is detected 70. This helps identify the pitch of the drive gear 40 which can be indicative of, and used to calculate, the movement of the drive nut 41.

As illustrated in FIG. 6, each of the plurality of magnets 64a, 64b, 64c, 64d, 64e, 64f, 64g, 64h, etc. produces a magnetic field identified as a red oval (2×) or a blue oval (1×) depending on the polarity of the magnet 64a, 64b, 64c, 64d, 64e, 64f, 64g, or 64h, etc. As the sensor 50 detects the edge transition 70 while the drive gear 40 rotates during operation of the medication delivery device, the sensor 50 detects areas in which a magnetic field is present 72 and detects areas in which a magnetic field 74 is not present.

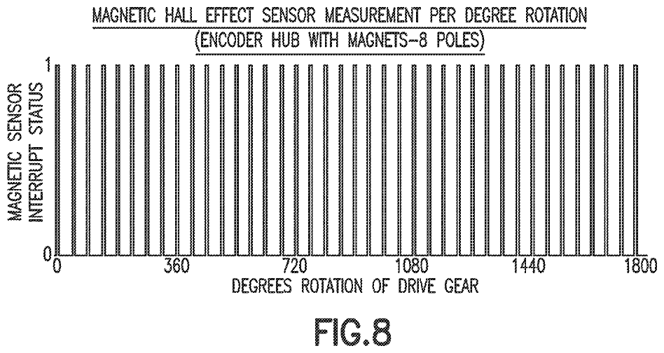

FIGS. 7 and 8 provide an illustrative example of this relationship in the chart showing the sensor output based on the angular rotational position of the magnetic encoder 60. When the magnetic field is sensed by the Hall Effect sensor 50, the sensor records a zero. On the other hand, when the magnetic field is not sensed by the Hall Effect sensor, the sensor records a one. As seen in FIGS. 7 and 8, the magnetic field is detected (sensor records zero) a majority of the time as the sensor 50 measures the magnetic field at the outer surface of the drive gear 40.

For example, magnetic Hall Effect sensor 50 can confirm drive gear 40 rotation without increasing friction acting on the drive gear 40 and can improve accuracy of operation since it does not introduce additional physical contact. Thus, the use of the magnetic Hall Effect sensor 50 can provide less physical impact, greater accuracy and a more power efficient gear operation resulting in enhanced battery life. Further, the magnetic Hall Effect sensor 50 can implement wireless communication, for example in cooperation with a Bluetooth device, for example, to facilitate seamless wireless communication with the medication delivery device 10 and external devices, such as the smart phone 52.

In another embodiment, the sensor 50 includes an analog sensor instead of a digital sensor. In this manner, the sensor 50 can measure a field strength, not simply 0 or 1.

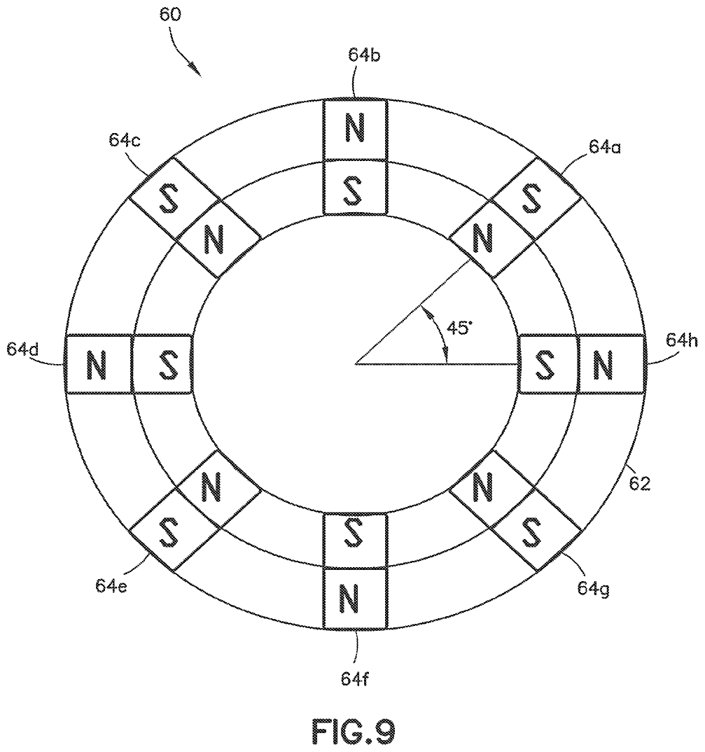

As illustrated in FIG. 9, the plurality of magnets 64a, 64b, 64c, 64d, 64e, 64f, 64g, 64h, etc. can be sufficiently spaced apart at approximately 45° apart to avoid overlap of the magnetic fields at the outer surface of the drive gear 40 where the edge transition 70 can be detected by the sensor 50. As a result, as illustrated in the example of FIGS. 7 and 8, there can be a clearer distinction when a magnetic field is detected 72 and when a magnetic field is not detected 74. On the other hand, if the plurality of magnets 64 are more narrowly spaced such that the magnetic fields overlap at the outer surface of the drive gear 40 where the edge transition 70 is detected by the sensor 50 magnetic fields may be identified irregularly such that the interrupts can be missed and a reduced number of switches may be observed by the magnetic Hall Effect sensor 50.

According to exemplary implementations of disclosed embodiments, the resolution of the magnetic encoder 60 can be varied and optimized using the magnetic Hall Effect sensor 50, for example, by increasing or decreasing the number of magnetic poles from the one or more magnets 64a, 64b, 64c, 64d, 64e, 64f, 64g, 64h, etc. observed with a given angular rotation so long as magnetic field interference or cross talk between the magnets is not problematic.

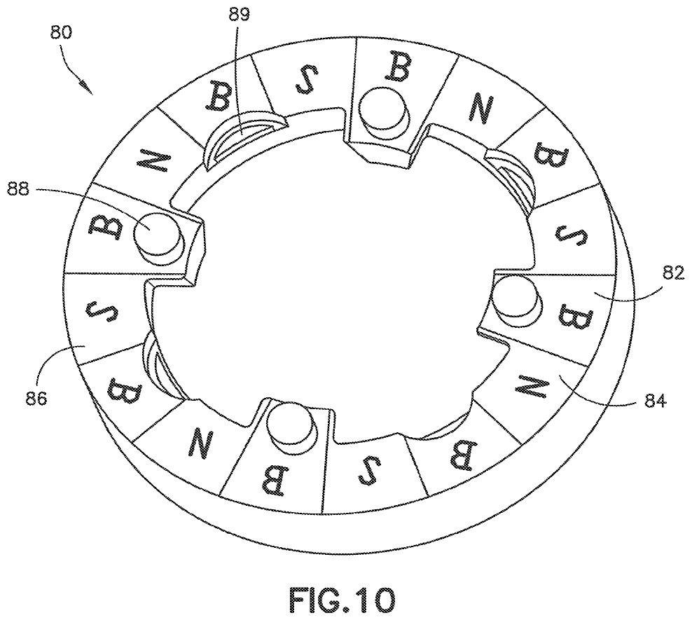

An example of FIG. 10 illustrates an alternate exemplary embodiment of the disclosure where a magnetic encoder 60 includes a magnet ring 80. For example, the magnet ring 80 can include a plurality of blank sections 82 and a plurality of magnetized sections. The plurality of magnetized sections can include a plurality of magnetic fields alternating north polarity 84 and south polarity 86. The blank sections 82 can be disposed between each magnet and each magnet can alternate in polarity in a circular direction. The blank sections 82 are not magnetized and can space out the magnets to control overlap of the magnetic fields from adjacent magnets. The plurality of magnets in the magnet ring 80 can be axially or radially polarized.

The blank sections 82 can further include a plurality of protrusions 88 and a plurality of semi-circular cavities 89. Each blank section 82 can include a protrusion 88 or a semi-circular cavity 89 in an alternating manner in a circular direction. The plurality of protrusions 88 and the plurality of semi-circular cavities 89 can be disposed on a top surface of the magnet ring 80.

In another embodiment, the magnet ring 80 can include no blank sections 82 and two magnetized sections. In this configuration, when two magnetized sections have opposite polarity, a bipolar detector can sense the rotation and function in a similar manner as described above.

An example of FIG. 11 illustrates an exemplary embodiment of the disclosure that provides a multi-pole magnet assembly 90 where a single piece magnet replaces the magnet member 64 and/or the encoder hub 62 of the exemplary embodiments described above. According to an exemplary embodiment, a single multi-pole magnet assembly 90 can include a plurality of magnet arms 92 disposed axially and evenly spaced in a circular direction. A single piece of magnetic material can be shaped into a desired form and polarized in slices around the multi-pole magnet assembly 90, for example, to simulate the presence of multiple individual magnets. In an exemplary implementation, the multi-pole magnet assembly 90 can include a central magnet and eight magnet arms 92.

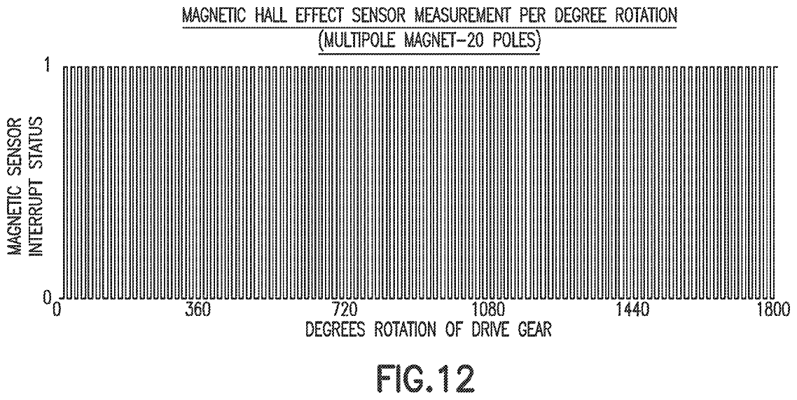

An example of FIG. 12 provides an illustrative chart showing the sensor output based on the angular rotational position of a multi-pole magnet assembly 90 having twenty poles. When comparing FIG. 8 and FIG. 12, according to an exemplary embodiment of the disclosure, configurations comprising more the magnetic poles can facilitate better resolution, provided that there is no magnetic field interference (overlap) or cross talk between magnets.

According to exemplary implementations, a single piece magnet of the multi-pole magnet assembly 90 can provide a low part count, increased manufacturability, reduced cost and a simpler production process. Further, being of a single piece magnetic material, the multi-pole magnet assembly 90 can be configured with more magnetic poles for a given size. In an exemplary implementation, multi-pole magnet assembly 90, as well as the magnetic encoder 60, can be configured without extra software control to operate via a lower power, continuously powered circuit resulting in power saving and possible longer battery life.

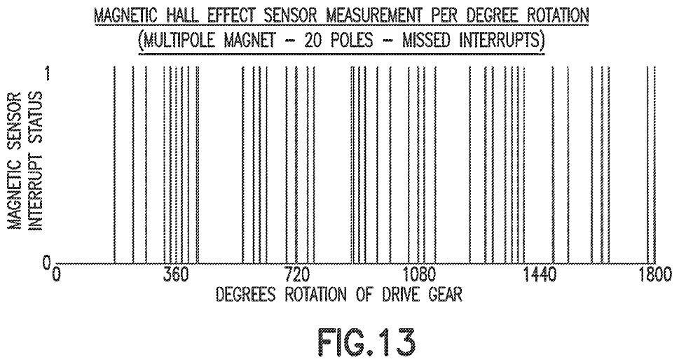

An example of FIG. 13 provides an illustrative chart showing where the magnetic field of adjacent magnet arms of the single multi-pole magnet assembly overlap. Such a configuration can result in many missed interrupts. The difference between FIGS. 12 and 13 illustratively shows the effects of overlapping magnetic fields or cross talking magnets. One possible factor for the overlapping magnetic fields is because the adjacent magnetic arms of a single multi-pole magnet assembly are too close to each other. Similarly, overlapping magnetic fields can also be experienced with a magnetic encoder and a ring magnet.

Other factors that can contribute to missed interrupts for a magnetic encoder, a ring magnet and a single multi-pole magnet assembly include a low magnetic field strength which can be based on the proximity and magnetic strength of the magnetized sections. For example, the greater the distance between the magnetized sections and a Hall Effect sensor, the lower a sensitivity of the magnetic field strength measurement. Also, the lower the magnetic strength of the magnetic arms, the lower the magnetic field strength. One or a combination of these factors can compromise a magnetic encoder, ring magnet and/or single multipole magnet assembly in such a manner that not all magnetic poles are detected.

It is to be understood that the example embodiments described herein can be subject to operative variations and alternative configurations to measure the dispensed medication. The components of the illustrative devices, systems and methods employed in accordance with the illustrated embodiments can be implemented, at least in part, in digital electronic circuitry, analog electronic circuitry, or in computer hardware, or in combinations of them. These components can be implemented, for example, as a machine-readable storage device, for execution by, or to control the operation of, data processing apparatus such as a processor, a computer, or multiple computers.

The various illustrative logical blocks, modules, and circuits described in connection with the embodiments disclosed herein may be implemented or performed with a general purpose processor, a digital signal processor (DSP), an ASIC, a FPGA or other logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, for example, a combination of a DSP and a microprocessor, a plurality of microprocessors, one or more microprocessors in conjunction with a DSP core, or any other such configuration.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, for example, magnetic, magneto-optical disks, or optical disks. Information carriers suitable for embodying computer program instructions and data include all forms of non-volatile memory, including by way of example, semiconductor memory devices, for example, electrically programmable read-only memory or ROM (EPROM), electrically erasable programmable ROM (EEPROM), flash memory devices, and data storage disks (for example, magnetic disks, internal hard disks, or removable disks, magneto-optical disks, and CD-ROM and DVD-ROM disks). The processor and the memory can be supplemented by, or incorporated in special purpose logic circuitry.

Those of skill in the art would understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those of skill would further appreciate that the various illustrative logical blocks, modules, circuits, and algorithm steps described in connection with the embodiments disclosed herein may be implemented as electronic hardware, for example. Various illustrative components, blocks, modules, circuits, and steps have been described above generally in terms of their functionality and may be implemented by hardware depending upon the particular application and design constraints imposed on the overall system. Skilled artisans may implement the described functionality in varying ways for each particular application, but such implementation decisions should not be interpreted as causing a departure from the scope of claims exemplified by the illustrative embodiments.

An exemplary storage medium is coupled to the processor such the processor can read information from, and write information to, the storage medium. In the alternative, the storage medium may be integral to the processor. In other words, the processor and the storage medium may reside in an integrated circuit or be implemented as discrete components. Computer-readable non-transitory media includes all types of computer readable media, including magnetic storage media, optical storage media, flash media and solid state storage media.

The above-presented description and figures are intended by way of example only and are not intended to limit the illustrative embodiments in any way except as set forth in the following claims. It is particularly noted that persons skilled in the art can readily combine the various technical aspects of the various elements of the various illustrative embodiments that have been described above in numerous other ways, all of which are considered to be within the scope of the claims.

What is claimed is:

1. A medication delivery device comprising:
a base plate engaging a cover to form an interior, the interior including:
a pump drive mechanism for driving medication through the device;
a drivetrain engaged to the pump drive mechanism;
a magnetic encoder including an encoder hub mechanically coupled to the drivetrain, the encoder hub including a magnet; and
a sensor that measures a magnetic field from the magnet to determine a rotational position of the drivetrain.

2. The medication delivery device of claim 1, wherein the medication delivery device includes one of a patch pump, an infusion pump and an automated insulin delivery device.

3. The medication delivery device of claim 1, wherein the pump drive mechanism includes a plunger driven by a leadscrew.

4. The medication delivery device of claim 3, wherein
the drivetrain includes a drive gear;
the drivetrain engages a drive nut; and
the drive nut is a final rotating component that engages the leadscrew.

5. The medication delivery device of claim 4, wherein the drive gear is aligned to the sensor.

6. The medication delivery device of claim 4, wherein the encoder hub is coupled to the drive gear to measure the rotational position.

7. The medication delivery device of claim 1, wherein the magnet includes a plurality of magnets evenly spaced around the encoder hub.

8. The medication delivery device of claim 7, wherein a polarity of the plurality of magnets facing away from a center axis of a drive gear remains constant.

9. The medication delivery device of claim 7, wherein a polarity of the plurality of magnets facing away from a center axis of a drive gear alternates in series with respect to the drive gear.

10. The medication delivery device of claim 1, wherein the magnet is axially polarized.

11. The medication delivery device of claim 1, wherein the magnet is radially polarized.

12. The medication delivery device of claim 1, wherein the magnet includes a single multi-pole magnet with a plurality of magnet arms equally spaced.

13. The medication delivery device of claim 1, wherein the sensor is mounted on a top surface of a circuit board.

14. The medication delivery device of claim 1, wherein the sensor is mounted on a bottom surface of a circuit board.

15. The medication delivery device of claim 4, wherein the sensor is aligned vertically with an axis of rotation of the drive gear.

16. The medication delivery device of claim 1, wherein the sensor includes one of a magnetic Hall Effect sensor and a magnetic resistance sensor.

17. The medication delivery device of claim 1, wherein the magnetic encoder includes a ring magnet having one or more magnetized sections.

18. A medication delivery device comprising:
a base plate engaging a cover to form an interior, the interior including:
a pump drive mechanism for driving medication through the device;
a drivetrain engaged to the pump drive mechanism; and
an optical sensor disposed with respect to the drivetrain;
wherein the optical sensor measures a rotational position of the drivetrain.

19. The medication delivery device of claim 18, further comprising an optical sensor disposed with respect to the drivetrain.

20. The medication delivery device of claim 19, wherein the optical sensor includes an emitter that emits light including a laser light and a receiver that receives the light.

21. The medication delivery device of claim 20, wherein
the optical sensor includes optical fan blades that are configured between an emitter and a receiver to split or block the light; and
the optical sensor is configured to detect when the light is split or blocked.
